(19) United States
(12) Patent Application Publication
(10) Pub. No.: US 2025/0299504 A1
(43) Pub. Date: Sep. 25, 2025

(54) Systems and Methods for Large-Scale Alignment of Tissue from Living to Postmortem

(71) Applicant: The Board of Trustees of the Leland Stanford Junior University, Stanford, CA (US)

(72) Inventors: Mark J. Schnitzer, Palo Alto, CA (US); Xiqian Jiang, Stanford, CA (US); Lun Wang, Stanford, CA (US)

(73) Assignee: The Board of Trustees of the Leland Stanford Junior University, Stanford, CA (US)

(21) Appl. No.: 19/084,579

(22) Filed: Mar. 19, 2025

Related U.S. Application Data
(60) Provisional application No. 63/567,377, filed on Mar. 19, 2024.
(60) Provisional application No. 63/774,602, filed on Mar. 19, 2025.

(51) Int. Cl. G06V 20/69; G06T 7/33; G06V 10/75; G06V 20/70; G16H 30/40

(52) U.S. Cl. CPC G06V 20/698; G06T 7/337; G06V 10/751; G06V 20/695; G06V 20/70; G16H 30/40; G06T 2200/04; G06T 2207/30016; G06T 2207/30024; G06V 2201/03

(57) ABSTRACT

Systems and methods for alignment of in vivo and ex vivo images in accordance with embodiments of the invention are illustrated. One embodiment includes a method for aligning in vivo with ex vivo captured tissue images, including obtaining a first image captured in vivo, obtaining a second image captured ex vivo, identifying cells in the first image and the second image, generating a soma-print for cells in the images, where each soma-print includes a plurality of vectors from a cell to each of its n nearest neighboring cells, computing a pair-wise soma-print score for each pairing of cells between the first plurality of cells and the second plurality of cells, identifying matched cell pairings between the first plurality of cells and the second plurality of cells based on their soma-print score, and annotating at least one of the images with the matched cell pairings.

### CROSS-REFERENCE TO RELATED APPLICATIONS

The current application claims the benefit of and priority under 35 U.S.C. § 119 (e) to U.S. Provisional Patent Application No. 63/567,377 entitled “Systems and Methods for Large-Scale Alignment of Tissue from Living to Postmortem” filed Mar. 19, 2024, and U.S. Provisional Patent Application No. 63/774,602 entitled “Systems and Methods for Large-Scale Alignment of Tissue from Living to Postmortem” filed Mar. 19, 2025. The disclosure of U.S. Provisional Patent Application Nos. 63/567,377 and 63/774,602 are hereby incorporated by reference in its entirety for all purposes.

### FIELD OF THE INVENTION

The present invention generally relates to aligning in vivo cellular recordings with post-mortem tissue samples.

### BACKGROUND

“Omics” refers to the scientific fields associated with measuring biological molecules. Cellular transcriptomics is a group of techniques that measure gene expression at different locations tissue. Spatial transcriptomics techniques have recently been used to localize cell types within tissue samples based on RNA expression. Cell-level tissue differentiation within a single sample has enabled a wide number of research breakthroughs and represents a leap forward in the omics revolution. Other “omics” fields include proteomics, connectomics, transcriptomics, and many others.

Neurons are a type of specialized cell that transmit electrical signals, called action potentials, across a network of neurons. This network of neurons is referred to as the nervous system, which includes the brain. The brain operates through complex neural circuits that process sensory information, execute movements, and support higher-order cognitive functions. The individual neurons that make up these circuits are not homogenous, and differ widely across various domains including gene expression profiles, connectivity patterns, and firing properties. The BRAIN Initiative Cell Census Network has identified over 300 major cell types and 5,000 transcriptionally distinct cell clusters in mouse brains.

### SUMMARY OF THE INVENTION

Systems and methods for alignment of in vivo and ex vivo images in accordance with embodiments of the invention are illustrated. One embodiment includes a method for aligning in vivo captured tissue images with ex vivo captured tissue images, including obtaining a first image captured in vivo of a tissue sample, obtaining a second image captured ex vivo of the tissue sample, identifying cells in the first image and the second image, generating a soma-print for a first plurality of cells in the first image and a second plurality of cells in the second image, where each soma-print includes a plurality of vectors from a cell to each of its n nearest neighboring cells, computing a pair-wise soma-print score for each pairing of cells between the first plurality of cells and the second plurality of cells, identifying matched cell pairings between the first plurality of cells and the second plurality of cells based on their soma-print score, and annotating at least one of the first image and the second image with the matched cell pairings.

In a further embodiment, the method further includes steps for generating a second soma-print for unmatched cells in the first plurality of cells and the second plurality of cells, where the second soma-print includes a plurality of vectors from the cell to each of its nearest matched neighboring cells, computing a second pair-wise soma-print score for each pairing of unmatched cells between the first plurality of cells and the second plurality of cells, identifying new matched cell pairings from the first plurality of cells and the second plurality of cells based on their second soma-print score, and annotating the at least one of the first image and the second image with the new matched cell pairings.

In still another embodiment, computing a pair-wise soma-print score between a first cell and a second cell includes matching each vector from a soma-print of the first cell to each vector from a soma-print of the second cell, sequentially identifying the two matched vectors having a smallest Euclidean distance at each step, and taking the average of the smallest Euclidean distance for each matched vector.

In a still further embodiment, the first image and the second image are three-dimensional images.

In yet another embodiment, the method further includes steps for obtaining the first image using an optical implant implanted into a live organism, extracting the tissue while maintaining an imaging plane of the optical implant with respect to the tissue, obtaining a slice of the tissue, where the slice of tissue is parallel to the imaging plane, and obtaining the second image by imaging the slice of tissue.

In a yet further embodiment, to extract the tissue while maintaining the imaging plane, the method further includes fixing the tissue in situ with the optical implant, where the optical implant creates a flat surface on the tissue in parallel with the imaging plane, extracting the tissue, embedding the tissue on a first plate using a second plate on an opposite side of the tissue from the first plate, where the first plate and second plate are parallel, and the embedding material and tissue create an embedded tissue block between the first plate and the second plate, creating a flat surface on a blank embedding material block parallel to a cutting plane of a cutting device, fixing the embedded tissue block to the blank embedding material block after removing at least one plate, and slicing the embedded tissue block using the cutting device.

In another additional embodiment, the optical implant is a gradient index lens.

In a further additional embodiment, the tissue is brain tissue, and the first plurality of cells and the second plurality of cells are neurons.

In another embodiment again, the method further includes steps for aligning the first image and the second image using prominent spatial landmarks prior to identifying cells in the images.

In a further embodiment again, the method further includes steps for annotating at least one of the first image and the second image with postmortem experimental data obtained using the second image.

In still yet another embodiment, the method further includes steps for warping at least one of the first image and the second image such that the first image and second image spatially align.

One embodiment includes a system for aligning in vivo captured tissue images with ex vivo captured tissue images, including a processor, and a memory containing an alignment application that configures the processor to obtain a first image captured in vivo of a tissue sample, obtain a second image captured ex vivo of the tissue sample, identify cells in the first image and the second image, generate a soma-print for a first plurality of cells in the first image and a second plurality of cells in the second image, where each soma-print includes a plurality of vectors from an cell to each of its n nearest neighboring cells, compute a pair-wise soma-print score for each pairing of cells between the first plurality of cells and the second plurality of cells, identify matched cell pairings between the first plurality of cells and the second plurality of cells based on their soma-print score, and annotate at least one of the first image and the second image with the matched cell pairings.

In a still yet further embodiment, the alignment application further configures the processor to generate a second soma-print for unmatched cells in the first plurality of cells and the second plurality of cells, where the second soma-print includes a plurality of vectors from the cell to each of its nearest matched neighboring cells, compute a second pair-wise soma-print score for each pairing of unmatched cells between the first plurality of cells and the second plurality of cells, identify new matched cell pairings from the first plurality of cells and the second plurality of cells based on their second soma-print score, and annotate the at least one of the first image and the second image with the new matched cell pairings.

In still another additional embodiment, to compute pair-wise soma-print scores between a first cell and a second cell, the alignment application further configures the processor to match each vector from a soma-print of the first cell to each vector from a soma-print of the second cell, sequentially identify the two matched vectors having a smallest Euclidean distance at each step, and take the average of the smallest Euclidean distance for each matched vector.

In a still further additional embodiment, the first image and the second image are three-dimensional images.

In still another embodiment again, the tissue is brain tissue, and the first plurality of cells and the second plurality of cells are neurons.

In a still further embodiment again, the alignment application further configures the processor to align the first image and the second image using prominent spatial landmarks prior to identifying cells in the images.

In yet another additional embodiment, the alignment application further configures the processor to annotate at least one of the first image and the second image with postmortem experimental data obtained using the second image.

In a yet further additional embodiment, the alignment application further configures the processor to warp at least one of the first image and the second image such that the first image and second image spatially align.

One embodiment includes a machine readable medium containing instructions that, when executed by a processor, configure the processor to perform the steps of obtaining a first image captured in vivo of a tissue sample, obtaining a second image captured ex vivo of the tissue sample, identifying cells in the first image and the second image, generating a soma-print for a first plurality of cells in the first image and a second plurality of cells in the second image, where each soma-print includes a plurality of vectors from an cell to each of its n nearest neighboring cells, computing a pair-wise soma-print score for each pairing of cells between the first plurality of cells and the second plurality of cells, identifying matched cell pairings between the first plurality of cells and the second plurality of cells based on their soma-print score, and annotating at least one of the first image and the second image with the matched cell pairings.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a flow chart for a tissue extraction process in accordance with an embodiment of the invention.

FIG. 2 is a graphical depiction of a tissue extraction process in accordance with an embodiment of the invention.

FIG. 3 is a flow chart for an alignment process in accordance with an embodiment of the invention.

FIG. 4 is a graphical depiction of a soma-print generation process in accordance with an embodiment of the invention.

FIG. 5 is a block diagram for an image alignment device in accordance with an embodiment of the invention.

### DETAILED DESCRIPTION

Measurement of cellular activity in living organisms is an important source of research data for many different fields of biology. For example, measurement of neural activity in live brain tissue has provided valuable insight around the structure and functioning of the nervous system, especially in connectomics. That said, there is a limit of what can be practically or ethically performed during an investigation with live tissue. Many techniques are available for the investigation of post-mortem tissue, and more are rapidly being developed. Spatial transcriptomics has recently emerged as a valuable technique for investigating tissue. While it is desirable to have measurements of how a cell (and its neighbors) acted when it was alive to compared to expression data extracted post-mortem, it has been nearly impossible to align 1:1 measurements of a live cell with measurements of the exact same cell post-mortem at scale and with high fidelity.

Attempts to solve the alignment problem have depended on sparse labeling, static red fluorescence as anchor points, and intrinsic features such as blood vessel landmarks in order to spatially align samples. Some attempts have also been made using computational reconstruction, which have relied upon immunohistochemical staining or genetically expressed fluorescent proteins to identify cell subtypes in tissue slices, and more recently on use of RNA fluorescence in situ hybridization (RNA FISH). However, these alignment techniques are often labor-intensive, operator-dependent, difficult to scale, and lack quantification, which limits applicability to high-throughput studies.

The primary challenge in alignment lies in the unpredictability of postmortem tissue handling. In particular, angular mismatches between regions of interest in vivo and ex vivo remain unresolved and largely depend on the precision of human operators, making consistent replication in large areas difficult. Furthermore, tissue anisotropy during various types of optical imaging exacerbates the challenge, rendering alignment in densely labeled areas nearly impossible. Additionally, pixel-level similarity-based computational alignment methods have also shown limited success in this context, partially because of the inconsistency of activity-dependent fluorescent signals between in vivo and ex vivo imaging, which will induce more difficulty when mixed with various mechanical and optical distortions.

A further challenge arises in classifying neurons based on connectivity. Traditional connectivity-tracing techniques, such as viral, chemical, or toxin-based fluorophores, suffer from limited color diversity, restrictions on co-labeling neurons with multiple projection targets, and potential toxicity in functional studies. A promising alternative is RNA barcode-based connectivity mapping, which integrates well with spatial transcriptomics and overcomes these challenges.

Systems and methods described herein address these decade old challenges using multimodal image processing and tissue extraction techniques that are scalable to high-throughput use cases. These systems and methods are collectively referred to as TRU-FACT (Total Registration Under Functional imaging, Awake behavior, Connectivity, and Transcriptomics), a high-throughput alignment method that integrates functional imaging, long-range connectivity mapping, and spatial transcriptomics to achieve precise, large-scale neural circuit mapping. Unlike previous approaches that rely on sparse labeling or computational reconstructions, TRU-FACT optimizes tissue processing and imaging to ensure high-fidelity alignment. By establishing reference planes in or out of the brain tissue that are parallel to the in vivo imaging plane—and employing curvature-matching or flat-field objectives—postmortem brain sections precisely aligned to the in vivo plane. Transferring of the parallel planes, real and virtual during tissue processing steps, creates a minimally altered tissue for any further analysis.

Additionally, in many embodiments, TRU-FACT incorporates projection barcoding and new algorithms for 2D and 3D image alignment with quantitative cell registration, making multimodal analysis more practical, efficient, and scalable. Soma-print, a vector-based near-neighbor high dimensional alignment algorithm described below, tolerates modest distortion and mismatch regardless of the source, allowing quantification and automatic alignment at large scale. This method is also compatible with various neural circuits and optical implants, broadening its applicability to a wide range of experimental paradigms.

In order to perform alignment, in vivo and postmortem ex vivo images from the same imaging plane must be obtained. Therefore, a discussion of processes for extracting and imaging tissue to enable alignment of images is found below, followed by a discussion of computational alignment processes.

### Tissue Extraction and Imaging

Imaging live tissue for research is often performed on model organisms, such as mice. While the below is described in the context of mouse brains, as one can appreciate, any model organism or tissue type can be studied using TRU-FACT. Direct investigation of live mouse brain tissue has been an invaluable research tool. In the last several decades, various approaches have been developed to gain direct optical access to live brain tissue including (but not limited to) utilization of gradient index (GRIN) lenses, Crystal Skulls, and prism probes. Many imaging modalities, including those listed above, involve an optical implant which is fixed with respect to the mouse brain. This provides a fixed field of view when imaging. The goal of tissue extraction is to maintain the field of view to subsequent ex vivo imaging steps.

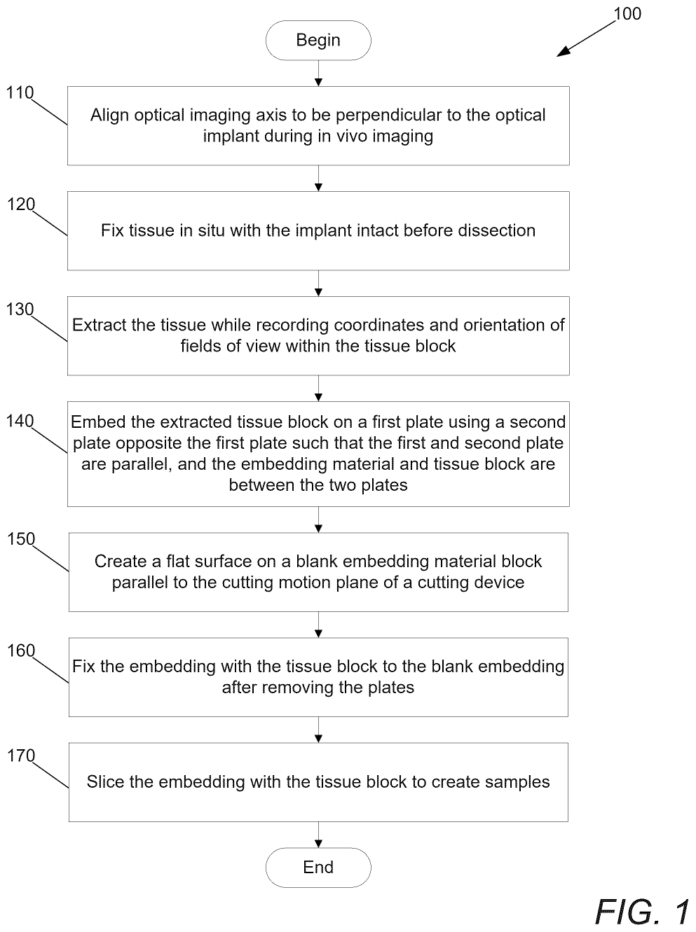

Turning now to FIG. 1, a process for extracting tissue while maintaining field of view in accordance with an embodiment of the invention is illustrated. Process 100 includes aligning (110) the optical imaging axis of the optical implant to be perpendicular to the optical implant during the in vivo imaging phase. Once imaging is completed and the subject is postmortem, the imaged tissue is fixed (120) in situ with the optical implant intact before dissection. In some embodiments, the tissue does not necessarily have to be fixed in situ. So long as the tissue is extracted in a trackable manner, tissue can be extracted as fresh, then frozen. In various embodiments, tissue could be transformed into a hydrogel embedding, then fixed and extracted. The tissue is then extracted (130) via dissection while maintaining a record of the coordinates and orientation of fields of view within the tissue block. In the case of a mouse brain, it is recommended to extract from the ventral side of the brain. Once extracted, the tissue is embedded (140) on a first plate in an embedding material. In numerous embodiments, the embedding material is agar, or optical cutting temperature (OCT) compound. The embedding is made such that the embedding block itself has two parallel surfaces, one formed along the first plate, and a second formed along a second plate parallel to the first plate. In order to make the first and second plates parallel (such that the embedding forms parallel surfaces), equal length pillars can be used between the two plates. In some embodiments, split plates are stacked up on either side of the embedding between the two plates, where one half of each plate is placed in order on either side, to form the pillars. However, premade pillars work as well as long as they are the same length. In some embodiments, the first plate is an optical viewing window from the optical implant.

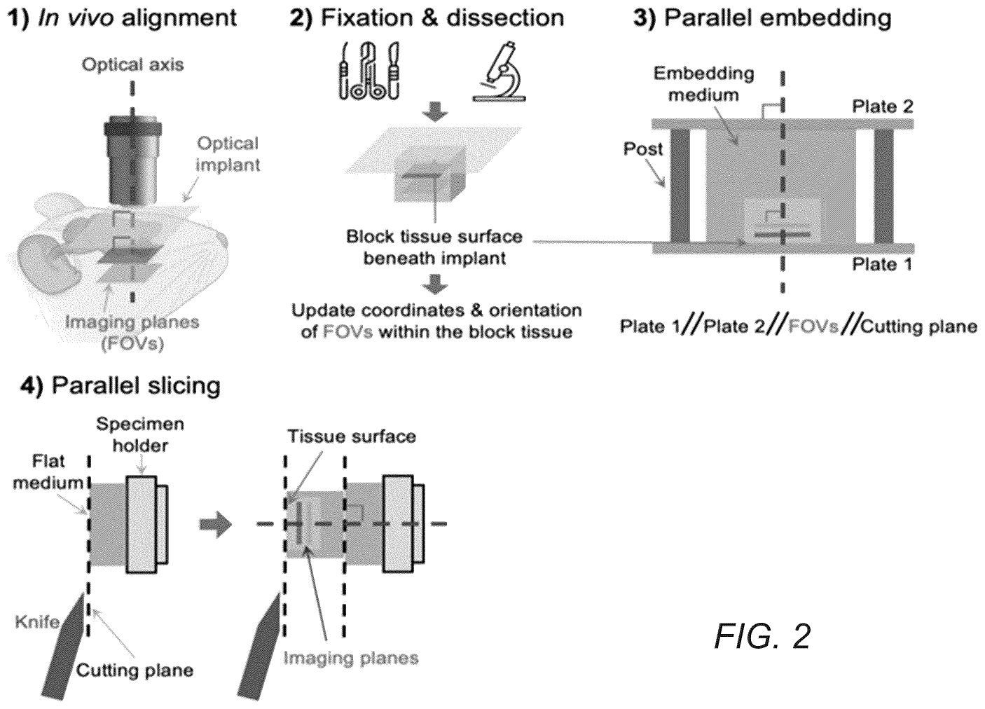

Separately, a blank embedding material block (e.g. an OCT block) is placed in a cutting device such as, but not limited to, a cryostat or a microtome, and trimmed parallel to the cutting plane. The tissue embedding is then fixed (160) to the blank after removing the plates such that only the embedding material and tissue remain in the device. Once fixed to the blank, the tissue can be sliced (170) to create samples for postmortem experimentation and imaging. This methodology preserves the fields of view from in vivo to ex vivo, and is graphically depicted in accordance with an embodiment of the invention in FIG. 2.

While a particular tissue extraction method is shown in FIGS. 1 and 2, other methods can be used to extract tissue while preserving fields of view. For example, a geometrically well defined object such as, but not limited to, an optical post or “ultra post”, can be implanted and fixed in the tissue block in order to provide a frame of reference for computationally determining fields of view in extracted tissue. In many embodiments, a reference object is attached to the optical implant of the chemically fixed tissue. In many embodiments, the reference object has one or more extremely flat surfaces. The post along with the optical implant and the sample are then fixed in a substance to form a cleavable block structure. The block is then cut to form a flat surface as described above. In many embodiments, the flat surface is parallel to the optical implant. This forms at least 3 reference planes (block surface, ultra post surface, and optical implant). The optical implant and post are then removed. The reference planes can then be used to generate a coordinate system for the entire tissue sample so the same cells identified during life can be identified postmortem.

As can be readily appreciated, any number of different tissue extraction methods can be used to obtain ex vivo slices that are parallel to the in vivo imaging plane as appropriate to the requirements of specific applications of embodiments of the invention. Once investigation and imaging is performed on the postmortem samples, alignment of in vivo images and images of the postmortem samples is performed using image processing techniques described below.

### Computational Image Alignment

In order to align images of in vivo and ex vivo images, systems and methods described herein identify spatial relationships between a cell and its neighbors within regions of interest. These spatial relationships are represented as a data structure containing n vectors from the center of the soma towards its nearest n neighboring somata, and is referred to as a soma-print. A soma-print score between two cells representing the similarity of their soma-prints is computed based on the average Euclidean distances of m corresponding vector pairs (msn, allowing potential mismatched neighboring cells). In many embodiments, m and n range between 5 and 30. In many embodiments, m is 10, and n is 15. However, these numbers can be modified by the user to suit the specific use case and application. For example, different types of tissue may benefit from different m and n ranges. As can be readily appreciated, these numbers can be modified without departing form the scope or spirit of the invention.

In numerous embodiments, the vectors from two cells are matched into pairs sequentially by identifying the two vectors with the smallest Euclidean distance at each step. The average Euclidean distances between paired vectors can then be converted to a score ranging from 0 to 100, where 100 corresponds to a distance of 0 reflective of maximum soma-print similarity. Soma-prints between two cells can be used to iteratively select the next “anchor” neighboring cells for the next soma-print to generate. By selecting matched cells with high scores (likelihood of a match<0.05), soma-print scores of cells with mismatched neighbors can be enhanced. That is, by iteratively selecting cells with high soma-print scores as anchors for the next iteration, by only computing n vectors towards only the neighboring cells that are already matched, better soma-prints can be generated, and therefore better soma-print scores and matches can be achieved in subsequent rounds. Soma-prints are not limited to 2-D and can be performed in 3-D by measuring the n-vectors across slices.

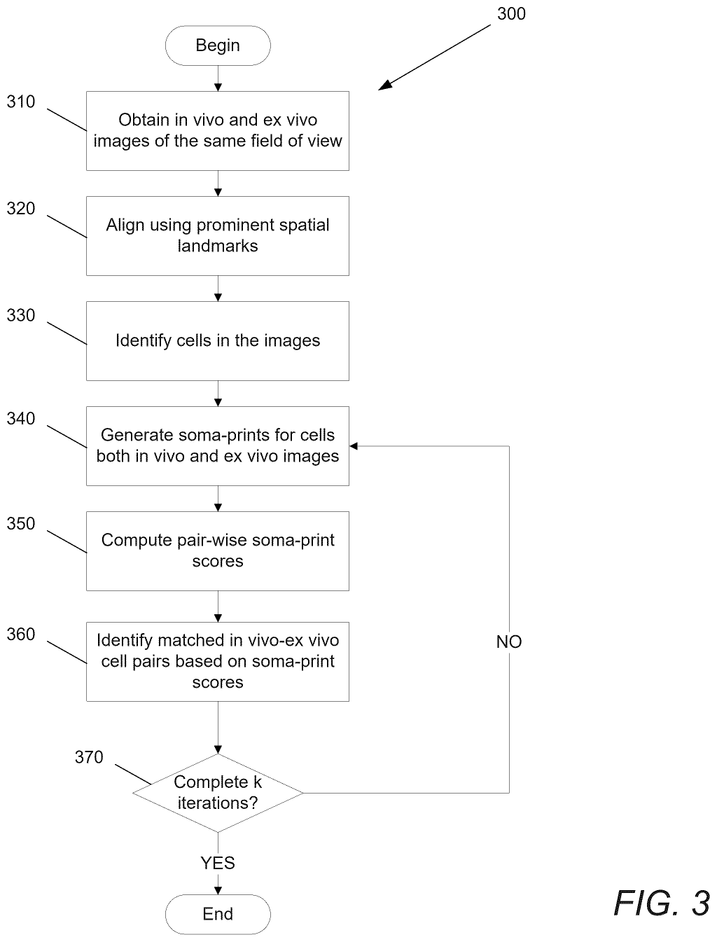

Soma-prints, as their name suggests, are valuable for aligning images because each soma-print is unique like a fingerprint. Turning now to FIG. 3, a process for aligning an in vivo and an ex vivo image using soma-prints in accordance with an embodiment of the invention is illustrated. Process 300 includes obtaining (310) an in vivo and an ex vivo image to be aligned, where the two images have the same field of view. In many embodiments, the same field of view is achieved using tissue extraction and slicing techniques described above. However, as long as the images have the same field of view, it is not relevant to the computational alignment process as to how that field of view was achieved. The images are aligned (320) based on any prominent spatial landmarks such as blood vessels. This alignment phase does not have to be accurate and does not identify cells in each image that specifically align. That is, this alignment roughly gets the two images into approximately the same orientation. However, this initial alignment phase is not mandatory, and in many embodiments is not performed, Assuming at least two matches can be found using soma-prints as described below, alignment of the image can be achieved by lining up the at least two identified cells. Individual cells in the two images are then identified (330) either manually, or more easily though existing cell segmentation methods. Once the individual cells are identified in the images, soma-prints are computed (340) for cells in both images. In many embodiments, between 5 and 30 closest neighbors are used per cell in the generation of the soma-print for a given cell (5$n$30 In numerous embodiments, every cell in each image has a soma-print computed. However, in various embodiments, only enough soma-prints across the two images needed to align all cells of interest are computed. In many embodiments, the registration of the cells to their match across images is an alignment as the two images can be one-to-one lined up. In some embodiments, the images themselves can be warped to line up when placed over one another.

Soma-prints between the in vivo image and the ex vivo image are compared by computing (350) soma-print scores for each in vivo-ex vivo cell permutation. Cells are then identified as matches (360) between the in vivo and ex vivo images based on the soma-print scores. In many embodiments, to identify matched in vivo-ex vivo cell pairs, the second best match scores of every in vivo cell are used to estimate the noise level. A Gaussian model is then used to fit the noise distribution: p₂(x)=N(x|μ, σ). The best match scores of every in vivo cell are then plotted as a potential match distribution and are fitted with a mixture Gaussian mode: p₁(x)=Σₖ₌₁^{K} πk. N(x|μₖ, σₖ), where K is the number of Gaussian components (K=2), and πₖ is the mixing coefficient of the k-th Gaussian, constrained such that the sum of all πₖ=1, and N(x|μₖ, σₖ) is the Gaussian distribution for component k. The likelihood ratio between the noise distribution and the potential match distribution is calculated as: likelihood ratio (x)=p₁(x)/p₂(x), and the cutoff is determined as the value where the likelihood ratio=0.05. Cell pairs with a soma-print score larger than the cutoff are determined to be a match.

While only one round of matching can be performed, additional iterations using previously matched cells for the next iteration of soma-print calculations reduce noise caused by mismatched cells. Therefore, if fewer than a required number of iterations have been performed (370), then a new iteration of soma-prints are computed (350). In some embodiments, the required number of iterations is a predetermined fixed number, e.g. 2-5. In various embodiments, the iterations are performed until all (or a desired number of) cells are matched. In various embodiments, both or either of a predetermined number or a threshold number of matched cells are used as a halting function on the number of iterations.

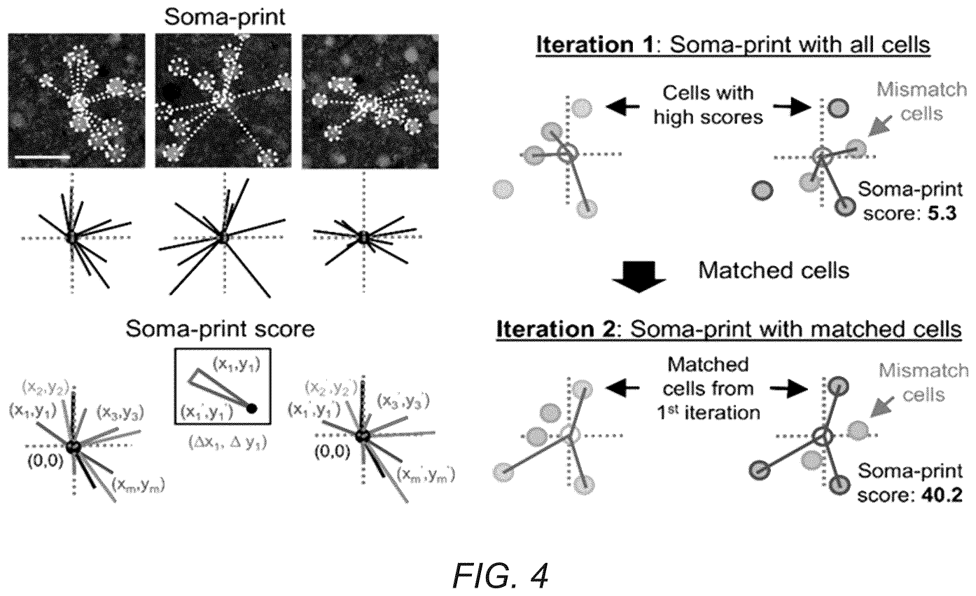

In these subsequent iterations, only cells previously matched are used as neighboring somata. In many embodiments, only unmatched cells have newly generated soma-prints. However, new soma-prints for all cells can also be calculated. These new soma-prints are then used to compute (350) new soma-print scores and identify (360) newly matched cells. At the end of the process, cells have been matched between the in vivo and ex vivo images, effectively registering all cells. The aligned images can then be used to merge data across different in vivo and ex vivo modalities. For ease of understanding, graphical representations of soma-prints, soma-print scores, and the iterative process in accordance with an embodiment of the invention are illustrated in FIG. 4.

In many embodiments, special considerations may be taken based on the imaging characteristics of the optical implant or ex vivo imaging optic. For example, imaging field curvature due to the nature of the optic may distort the image making alignment more difficult. Field curvature can be characterized by taking z-stack imaging of a fluorescent plate placed perpendicular to the optical axis. The z-stack imaging is taken across the optical focal plane. For imaging using either two-photon microscopy or GRIN lenses, the z-stack images can be processed first by cropping 6 pixels from each side to create 500×500 pixels images. For the large FOV, the image can be kept at 2048×2048 pixels images. Then, a gaussian fit can be applied at each pixel along the axial direction to determine the depths with peak intensity through the center of its fit. The fitting parameters can be initialized with boundaries that allow it to make a fit within the axial range of the z-stack imaging. The cropping of the image in the first step avoids the difficulty of fitting due to large noise along the edges of the image planes. Finally, the depth difference from the bottom of the field curvature can be calculated by the difference between the average of pixel depths within the 20 μm radius at the center of the image and the averages of pixel depths at the same radial distances from the center. The error values are computed from the standard deviations when mean values are calculated, and can be used to flatten images produced by the measured optic. As can be readily appreciated, optical distortions can be measured in any of a variety of different ways to preprocess images for subsequent alignment. Various extended applications of TRU-FACT are discussed below.

### Example Applications

An advantage of alignment between in vivo and ex vivo is molecular characterization of individual neurons measured in vivo. However, mere alignment does not provide information about long-range connectivity properties alone. To address this, barcoded viral tracers can be combined with multiround multiplexed hybridization chain reaction fluorescence in situ hybridization (HCR-FISH) imaging, enabling mapping of projection patterns while simultaneously identifying molecular and functional properties. By integrating these tools, for a given specific set of neurons, connectivity can be measured in vivo, their subtypes based on gene expression measured ex vivo, and their circuit properties linked to activity patterns during live behavior.

To design the barcodes, 100-200 bp optimized sequences based on annealing efficiency and exclusion of homologous host genome sequences are generated and packaged into a retrograde AAV vector. Experimentally, at least eight distinct barcodes can be simultaneously expressed and detected within single cells. Different barcodes can be injected into different areas of the brain and used to trace long-range connectivity.

Even without barcoding, many different ex vivo and in vivo measurements can provide increased information when aligned. For example, Ca²⁺ imaging can be aligned with spatial transcriptomics information such as (but not limited to) HCR-FISH, MERFISH, CODEX, or any other imaging-based measurement technique as appropriate to the requirements of specific applications of embodiments of the invention.

### Processing Platforms

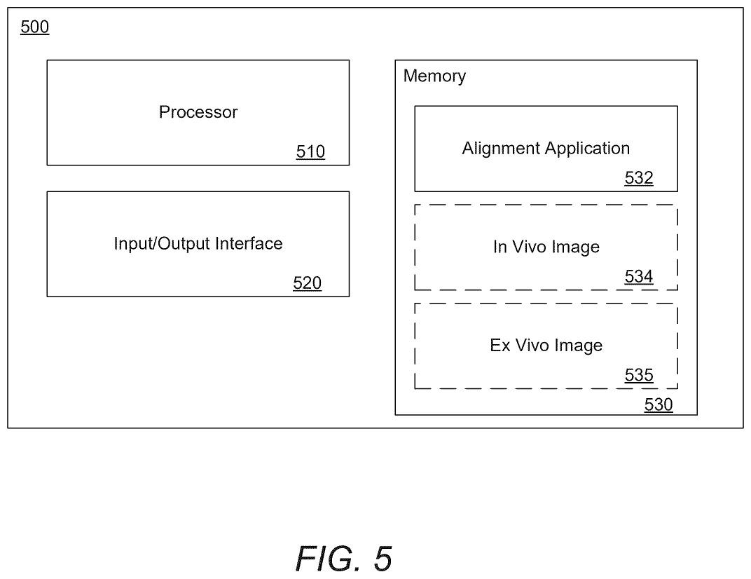

Turning now to FIG. 5, a block diagram for an alignment device capable of performing alignment processes described above is illustrated. Processing device 500 includes a processor 510. Processors can be any processing circuitry including (but not limited to) central processing units, graphics processing units, application-specific integrated circuits, field-programmable gate arrays, and/or any other logic circuit or combination thereof. The processing device 500 further includes an input/output (I/O) interface 520. I/O interfaces are capable of obtaining information for the processing device and transmitting information from the processing device. For example, an I/O interface may be used to communicating with displays or input devices. Further, I/O interfaces may be capable of wired and/or wireless communication over a network.

The processing device 500 further includes a memory 530. The memory may be volatile memory, nonvolatile memory, or a combination thereof. The memory 530 stores an alignment application that contains instructions that configure the processor to execute alignment processes described herein. In many embodiments, the memory 530 at various times stores in vivo 534 and ex vivo 536 images. The in vivo and ex vivo images may be annotated with metadata that identifies the matched cells in the counterpart image. As can be readily appreciated, any number of different computing platforms including (but not limited to) servers, personal computers, transcriptomics devices, and/or any other computational device can be made to align images as appropriate to the requirements of specific applications of embodiments of the invention.

Although specifics are discussed above, many different alignment methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for aligning in vivo captured tissue images with ex vivo captured tissue images, comprising:
obtaining a first image captured in vivo of a tissue sample;
obtaining a second image captured ex vivo of the tissue sample;
identifying cells in the first image and the second image;
generating a soma-print for a first plurality of cells in the first image and a second plurality of cells in the second image, where each soma-print comprises a plurality of vectors from a cell to each of its n nearest neighboring cells;
computing a pair-wise soma-print score for each pairing of cells between the first plurality of cells and the second plurality of cells;
identifying matched cell pairings between the first plurality of cells and the second plurality of cells based on their soma-print score; and
annotating at least one of the first image and the second image with the matched cell pairings.

2. The method of claim 1, further comprising:
generating a second soma-print for unmatched cells in the first plurality of cells and the second plurality of cells, where the second soma-print comprises a plurality of vectors from the cell to each of its nearest matched neighboring cells;
computing a second pair-wise soma-print score for each pairing of unmatched cells between the first plurality of cells and the second plurality of cells;
identifying new matched cell pairings from the first plurality of cells and the second plurality of cells based on their second soma-print score; and
annotating the at least one of the first image and the second image with the new matched cell pairings.

3. The method of claim 1, wherein computing a pair-wise soma-print score between a first cell and a second cell comprises:
matching each vector from a soma-print of the first cell to each vector from a soma-print of the second cell;
sequentially identifying the two matched vectors having a smallest Euclidean distance at each step; and
taking the average of the smallest Euclidean distance for each matched vector.

4. The method of claim 1, wherein the first image and the second image are three-dimensional images.

5. The method of claim 1, further comprising:
obtaining the first image using an optical implant implanted into a live organism;
extracting the tissue while maintaining an imaging plane of the optical implant with respect to the tissue;
obtaining a slice of the tissue, where the slice of tissue is parallel to the imaging plane; and
obtaining the second image by imaging the slice of tissue.

6. The method of claim 5, wherein to extract the tissue while maintaining the imaging plane, the method further comprises:
fixing the tissue in situ with the optical implant, where the optical implant creates a flat surface on the tissue in parallel with the imaging plane;
extracting the tissue;
embedding the tissue on a first plate using a second plate on an opposite side of the tissue from the first plate, where the first plate and second plate are parallel, and the embedding material and tissue create an embedded tissue block between the first plate and the second plate;
creating a flat surface on a blank embedding material block parallel to a cutting plane of a cutting device;
fixing the embedded tissue block to the blank embedding material block after removing at least one plate; and
slicing the embedded tissue block using the cutting device.

7. The method of claim 6, wherein the optical implant is a gradient index lens.

8. The method of claim 1, wherein the tissue is brain tissue, and the first plurality of cells and the second plurality of cells are neurons.

9. The method of claim 1, further comprising aligning the first image and the second image using prominent spatial landmarks prior to identifying cells in the images.

10. The method of claim 1, further comprising annotating at least one of the first image and the second image with postmortem experimental data obtained using the second image.

11. The method of claim 1, further comprising warping at least one of the first image and the second image such that the first image and second image spatially align.

12. A system for aligning in vivo captured tissue images with ex vivo captured tissue images, comprising:
a processor; and
a memory containing an alignment application that configures the processor to:
obtain a first image captured in vivo of a tissue sample;
obtain a second image captured ex vivo of the tissue sample;
identify cells in the first image and the second image;
generate a soma-print for a first plurality of cells in the first image and a second plurality of cells in the second image, where each soma-print comprises a plurality of vectors from an cell to each of its n nearest neighboring cells;
compute a pair-wise soma-print score for each pairing of cells between the first plurality of cells and the second plurality of cells;
identify matched cell pairings between the first plurality of cells and the second plurality of cells based on their soma-print score; and
annotate at least one of the first image and the second image with the matched cell pairings.

13. The system of claim 12, wherein the alignment application further configures the processor to:
generate a second soma-print for unmatched cells in the first plurality of cells and the second plurality of cells, where the second soma-print comprises a plurality of vectors from the cell to each of its nearest matched neighboring cells;
compute a second pair-wise soma-print score for each pairing of unmatched cells between the first plurality of cells and the second plurality of cells;
identify new matched cell pairings from the first plurality of cells and the second plurality of cells based on their second soma-print score; and
annotate the at least one of the first image and the second image with the new matched cell pairings.

14. The system of claim 12, wherein to compute pair-wise soma-print scores between a first cell and a second cell, the alignment application further configures the processor to:
match each vector from a soma-print of the first cell to each vector from a soma-print of the second cell;
sequentially identify the two matched vectors having a smallest Euclidean distance at each step; and
take the average of the smallest Euclidean distance for each matched vector.

15. The system of claim 12, wherein the first image and the second image are three-dimensional images.

16. The system of claim 12, wherein the tissue is brain tissue, and the first plurality of cells and the second plurality of cells are neurons.

17. The system of claim 12, wherein the alignment application further configures the processor to align the first image and the second image using prominent spatial landmarks prior to identifying cells in the images.

18. The system of claim 12, wherein the alignment application further configures the processor to annotate at least one of the first image and the second image with postmortem experimental data obtained using the second image.

19. The system of claim 12, wherein the alignment application further configures the processor to warp at least one of the first image and the second image such that the first image and second image spatially align.

20. A machine readable medium containing instructions that, when executed by a processor, configure the processor to perform the steps of:
obtaining a first image captured in vivo of a tissue sample;
obtaining a second image captured ex vivo of the tissue sample;
identifying cells in the first image and the second image;
generating a soma-print for a first plurality of cells in the first image and a second plurality of cells in the second image, where each soma-print comprises a plurality of vectors from an cell to each of its n nearest neighboring cells;
computing a pair-wise soma-print score for each pairing of cells between the first plurality of cells and the second plurality of cells;
identifying matched cell pairings between the first plurality of cells and the second plurality of cells based on their soma-print score; and
annotating at least one of the first image and the second image with the matched cell pairings.
